(19) United States
(12) Patent Application Publication
(10) Pub. No.: US 2026/0265668 A1
(43) Pub. Date: Sep. 10, 2026

(54) CELL CULTURE PROCESSES

(71) Applicant: UCB BIOPHARMA SRL, BRUSSELS (BE)

(72) Inventors: BASSEM BEN YAHIA, BRUSSELS (BE); THOMAS ARISTIDE DAHOMAIS, BRUSSELS (BE); STEFANIE J. M. EGGERMONT, BRUSSELS (BE); ANTOINE PHILIPPE THOMAS PIEDNOIR, BRUSSELS (BE)

(21) Appl. No.: 18/875,239

(22) PCT Filed: Jun. 14, 2023
(86) PCT No.: PCT/EP2023/065906

§ 371 Date: Dec. 16, 2024

(30) Foreign Application Priority Data
Jun. 15, 2022 (GB) 2208782.9
Oct. 25, 2022 (GB) 2215798.6
Jan. 20, 2023 (GB) 2300877.4

(51) Int. Cl. C12M 1/34; C12M 1/42; C12N 5/071; C12N 15/79

(52) U.S. Cl. CPC C12M 41/32; C12M 35/00; C12N 5/0682; C12N 15/79; C12N 2510/04

(57) ABSTRACT

The present invention belongs to the field of the manufacture of recombinant proteins, particularly antibodies. More specifically, it relates to methods of producing recombinant proteins (such as antibodies) in a bioreactor and/or of increasing cell culture performance during the production of recombinant protein in bioreactors (N stage) via specific feeding strategy in the seed bioreactor (N−1 stage), namely by applying organised, operational stress during seed culture by increasing feeding speed, feeding quantity and/or increasing power input via impellers.

### FIELD OF INVENTION

The present invention belongs to the field of the manufacture of recombinant proteins, particularly antibodies. More specifically, it relates to methods of producing recombinant proteins (such as antibodies) in a bioreactor and/or of increasing cell culture performance during the production of recombinant protein in bioreactors (N stage) via specific feeding strategy in the seed bioreactor (N−1 stage).

### BACKGROUND OF THE INVENTION

Development of recombinant proteins as therapeutic proteins, such as therapeutic antibodies, requires production of the recombinant proteins at an industrial scale. In order to achieve this, different expression systems, both prokaryotic and eukaryotic systems, may be employed. Over the past two decades, however, the majority of the therapeutic proteins approved as therapeutic have been manufactured through mammalian cell cultures and such system remains the preferred expression system for producing large quantity of recombinant proteins for human use.

Over the last 30 years, much effort has been dedicated to establishing the basic parameters of cell culture and recombinant protein expression with much focus of the research dedicated to reaching optimal cell growth through changes of the composition of the cell culture media (see e.g. Hecklau C. et al., 2016; Zang Li. et al., 2011) and operating conditions and, development of large bioreactors.

It is known that the performance of the production step (N stage) can be strongly impacted by the N−1 stage conditions. As described by Grammatikos et al (1999) the relative intracellular content of nucleotide and ATP, for instance, are impacted by the bioreactor conditions and especially by the seed bioreactor conditions.

Yet, there remains the need to provide N−1 stage cell culture methods which will positively impact the N stage of production of recombinant proteins.

### SUMMARY OF THE INVENTION

In a first aspect, the invention provides a process for producing a recombinant protein in a production bioreactor, wherein the process comprises: a. inoculating a N−1 bioreactor with mammalian cells comprising a gene that encodes the recombinant protein; b. culturing the mammalian cells in the N−1 bioreactor run in a fed-batch mode under specific conditions selected from: i. specific modes and duration of addition of the feed or of at least one of the feeds, ii. control of the total quantity of feed(s) to be added, and/or iii. control of at least one engineering parameter; c. inoculating a N bioreactor at a seeding density of at least 2.00×10⁶ viable cells/ml with cells obtained from step (b); d. culturing the cells in the N bioreactor under conditions that allow production of the recombinant protein, e. and optionally harvesting the recombinant protein, purifying the recombinant protein and formulating the recombinant protein.

In a second aspect, the invention provides a process for improving mammalian cell growth in a production bioreactor wherein the process comprises: a. inoculating a N−1 bioreactor with mammalian cells comprising a gene that encodes the recombinant protein; b. culturing the mammalian cells in the N−1 bioreactor run in a fed-batch mode under specific conditions selected from: i. specific modes and duration of addition of the feed or of at least one of the feeds, ii. the control of the total quantity of feed(s) to be added, and/or iii, the control of at least one engineering parameter; c. inoculating a N bioreactor at a seeding density of at least 2.00×10⁶ viable cells/ml with cells obtained from step (b); d. culturing the cells in the N bioreactor under conditions that allow production of the recombinant protein, e. and optionally harvesting the recombinant protein, purifying the recombinant protein and formulating the recombinant protein.

In a third aspect, the invention relates to a process for increasing the yield of production of a recombinant protein expressed by mammalian cells in culture in a production bioreactor, wherein the process comprises: a. inoculating a N−1 bioreactor with mammalian cells comprising a gene that encodes the recombinant protein; b. culturing the mammalian cells in the N−1 bioreactor run in a fed-batch mode under specific conditions selected from: i. specific modes and duration of addition of the feed or of the feed or of at least one of the feeds, ii. control of the total quantity of feed(s) to be added, and/or iii. control of at least one engineering parameter; c. inoculating a N bioreactor at a seeding density of at least 2.00×10⁶ viable cells/ml with cells obtained from step (b); d. culturing the cells in the N bioreactor under conditions that allow production of the recombinant protein, e. and optionally harvesting the recombinant protein, purifying the recombinant protein and formulating the recombinant protein.

### Definitions

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

As used in the specification and claims, the term “and/or” used in a phrase such as “A and/or B” herein is intended to include “A and B”, “A or B”, “A”, and “B”.

As used in the specification and claims, the term “cell culture” or “culture” is meant the growth and propagation of cells in vitro, i.e. outside of an organism or tissue. Suitable culture conditions for mammalian cells are known in the art, such as taught in Ozturk & Hu (2005). Mammalian cells may be cultivated in suspension or while attached to a solid substrate.

The terms “cell culture medium,” “culture medium”, “medium,” and any plural thereof, refer to any medium in which cells of any type can be cultivated. A “basal medium” refers to a cell culture medium that contains all of the essential ingredients useful for cell metabolism. This includes for instance amino acids, lipids, carbon source, vitamins and mineral salts. DMEM (Dulbeccos' Modified Eagles Medium), RPMI (Roswell Park Memorial Institute Medium) or medium F12 (Ham's F12 medium) are examples of commercially available basal media. Other suitable media have been described for instance in WO98/08934 and US2006/0148074 (both incorporated herein in their entirety). Further suitable commercially available media include, but are not limited to, AmpliCHO CD medium, Dynamis™ Medium, EX-CELL® Advanced™ CHO Fed-batch System, CD FortiCHO™ medium, CP OptiCHO™ medium, Minimum Essential Media (MEM), BalanCD® CHO Growth A Medium, ActiPro™ medium, DMEM-Dulbecco's Modified Eagle Medium and RPMI-1640 medium. Alternatively, said basal medium can be a proprietary medium, also herein called “chemically defined medium” or “chemically defined culture medium”, in which all of the components can be described in terms of the chemical formulas and are present in specific concentrations. The culture medium is preferably free of proteins and free of serum and can be supplemented by any additional compound(s) such as amino acids, salts, sugars, vitamins, hormones, growth factors, depending on the needs of the cells in culture.

The term “feed medium” (and plural thereof) refers to a medium used as a supplementation during culture, in fed-batch mode, to replenish the nutrients which are consumed during the culture. The feed medium can be a commercially available feed medium or a proprietary feed medium. Suitable commercially available feed media include, but are not limited to, Cell Boost™ supplements, EfficientFeed™ supplements, ExpiCHO™ Feeds. Alternatively, said feed medium can be a proprietary feed medium, also herein called “defined feed medium” or “chemically defined feed medium”, in which all of the components can be described in terms of the chemical formulas and are present in specific concentrations. A feed medium is typically concentrated in order not to increase to a high level the total volume of the culture in a bioreactor. Such a feed medium can contain most of the components at, for example, about 1.5×, 2×, 5×, 6×, 7×, 8×, 9×, 10×, 12×, 14×, 16×, 20×, 30×, 50×, 100×, 200× or even 500× of their normal amount in a basal medium. Proprietary feed media are typically in powder. Commercial feeds are either liquid or in powder. When feeds are already in liquid form, they are typically used as such, according to the leaflet. Feeds which are in powder need to be solubilised, in water for instance, before use.

Different feed media of different compositions can be added throughout the culture process. For instance, three different feed media can be used during the same process: one feed medium comprising most of the nutrients which are consumed (this feed is also named main feed medium), one feed medium comprising some further nutrients for instance when these nutrients present aggregation/stability issues (this feed is also named secondary feeds) and one feed medium comprising or consisting of the carbon source (e.g. glucose). It is noted that the secondary feed(s) are not mandatory and will depend on the components contained in the main feed. It may happen that only two feeds are used (i.e. the main feed and a feed comprising the carbon source) or it may also happen that only one main feed comprising all the components that are needed (including the carbone source) is used. It should be understood that in the case where only one feed is used in the context of the invention it can be referred to indifferently as “the feed” or “the main feed”. The term “bioreactor” refers to any system in which cells can be cultivated. It includes but is not limited to flasks, static flasks, spinner flasks, tubes, shake tubes, shake bottles, wave bags, bioreactors, fibre bioreactors, and stirred-tank bioreactors with or without microcarriers. Alternatively, this term also includes microtiter plates, capillaries or multi-well plates. Any size of bioreactor can be used, for instance from 1 millilitre (1 mL, very small scale) to 20000 litres (20000 L or 20 KL, very large scale), such as 1 mL, 5 mL, 0.01 L, 0.1 L, 1 L, 2 L, 5 L, 10 L, 50 L, 100 L, 500 L, 1000 L (or 1 KL), 2000 L (or 2 KL), 5000 L (or 5 KL), 10000 L (or 10 KL), 15000 L (or 15 KL) or 20000 L (20 KL).

The term “fed-batch culture” refers to a method of culturing cells, where there is a bolus (typically several bolus) or continuous feed medium (or feed media) supplementation to replenish the nutrients which are consumed, without removal of any medium already in the bioreactor. Feed(s) can be added according to a predetermined schedule of, for example, every day, once every other day, once every three days, etc. Alternatively, should the feeding be continuous, the feeding rate can be varied throughout the culture. This cell culture technique has the potential to obtain high cell densities in the order of greater than 8×10⁶ to 30×10⁶ cells/ml, depending on the media formulations, cell line, and other cell growth conditions. A biphasic culture condition can be created and sustained by a variety of feed strategies and media formulations.

The term “N−1 stage” according to the present invention corresponds to the stage just before the “production stage” during which cells are deemed to grow and multiply quickly (in a so-called seed bioreactor) in order to have enough material to inoculate the N stage bioreactor. Alternatively this stage is called seed stage.

The terms “production phase”, “production stage” or “N stage” according to the present invention correspond to the stage of cell culturing during the process for manufacturing a recombinant protein when the cells express (i.e. produce) the recombinant polypeptide(s). The production phase begins when the titre of the desired product increases and ends with harvest of the cells or the cell culture fluid or supernatant. Typically, at the beginning of the production phase, the cell culture is transferred from a seed bioreactor to a production bioreactor. Harvest is the step during which the cell culture fluid is removed from the production bioreactor, in order for the recombinant protein e.g. the recombinant antibody, to be recovered and purified in subsequent steps.

As used herein, “cell concentration” (also known as “cell density”) refers to the number of cells in a given volume of culture medium.

The term “Viable cell concentration” (or “VCC”) refers to the number of living cells in a given volume of culture medium. This is determined by standard viability assays. It should be understood that the skilled person knows how to determine the maximum VCC for each specific cell line: this is typically performed thanks to one or more initial experiments. Alternatively it may be referred to “VCD” or “viable cell density”.

The term “IVCC” refers to the integral viable cell count and can be determined by finding the area under the cell culture growth curve

$$( IVCC = {\int }_{0}^{t} VCC * dt ) .$$

The terms “viability” or “cell viability” refer to the ratio between the total number of viable cells and the total number of cells in culture. Although the viability is typically acceptable as long as it does not go below a 60% threshold compared to the start of the culture, the acceptable threshold can be determined on a case-by-case basis. Viability is often used to determine time for harvest (this determination is done once for all during preliminary experiments). For instance, in fed-batch culture, harvest can be performed once viability reaches at least 60% or after about 14 days (typically 14 days+/−1 day) in culture. Standard methods can be used to determine the cell viability (alternatively VCC or VCD), such as via the use of the VI-CELL® XR automated cell counting device (Beckman-Coulter Inc.).

The wording “titre” refers to the concentration of the protein of interest in a given volume of solution. This is determined by standard titre assays, such as serial dilutions combined with a detection method (colorimetric, chromatographic etc.), with a CEDEX or protein A high-pressure liquid chromatography (HPLC), Biacore C® or ForteBIO Octet® methods, as used in the example section.

The term “specific productivity”, also known as “qp”, refers to the amount of protein of interest, produced per cell per day.

The term “higher titre” or “higher productivity”, and equivalents thereof, means that the titre or the productivity is increased by at least 10% when compared to the control culture condition. The titre or specific productivity will be considered as maintained if it is in the range of −10% to 10% compared to the control culture condition. The terms “lower titre” or “lower productivity”, and equivalents thereof, means that the titre or the productivity is decreased by at least 10% when compared to the control culture condition.

The term “lag phase” refers to a period of slow growth when the cells are adapting to the culture environment and preparing for fast growth.

The term “specific power input” describes the ratio between the power input (P) and the volume of working fluid (V). The power input describes the quantity of energy delivered by the impellers to the bulk of the bioreactor per second. The specific power input is expressed as follows:

$$\frac{P}{V} = \frac{{N}_{p} \cdot ρ \cdot {( \frac{N}{60} )}^{3} {d}^{5}}{V}$$

### Where:

Nₚ is the power number, several examples of the power number are reported in the literature (Roustan, 2005);
ρ is the density of fluid (kg/m³);
N is the mixing speed (rpm);
d is the diameter if the impeller (m);
V is the working volume (m³).

The term “protein” as used herein includes peptides, polypeptides and proteins and refers to compound comprising two or more amino acid residues. A protein according to the present invention includes but is not limited to a cytokine, a growth factor, a hormone, a fusion protein, an antibody or a fragment thereof. A therapeutic protein refers to a protein that can be used or that is used in therapy.

The term “recombinant protein” means a protein produced by recombinant technics. Recombinant technics are well within the knowledge of the skilled person (see for instance Sambrook et al., 1989, and updates).

Preferably, the protein according to the methods, uses and processes of the present invention is an antibody or antigen-binding fragment thereof or a fusion protein.

The term “antibody” as used herein includes, but is not limited to, monoclonal antibodies, polyclonal antibodies and recombinant antibodies that are generated by recombinant technologies as known in the art. “Antibody” include antibodies of any species, in particular of mammalian species; such as human antibodies of any isotype, including IgG1, IgG2a, IgG2b, IgG3, IgG4, IgE, IgD and antibodies that are produced as dimers of this basic structure including IgGA1, IgGA2, or pentamers such as IgM and modified variants thereof; non-human primate antibodies, e.g. from chimpanzee, baboon, rhesus or cynomolgus monkey; rodent antibodies, e.g. from mouse, or rat; rabbit, goat or horse antibodies; camelid antibodies (e.g. from camels or llamas such as Nanobodies™) and derivatives thereof; antibodies of bird species such as chicken antibodies; or antibodies of fish species such as shark antibodies. The term “antibody” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old-World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody properties. Humanization reduces the immunogenicity of non-human antibodies in humans, thus facilitating the application of antibodies to the treatment of human disease. Humanized antibodies and several different technologies to generate them are well known in the art. The term “antibody” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody. The term “antibody” refers to both glycosylated and aglycosylated antibodies. Furthermore, the term “antibody” as used herein not only refers to full-length antibodies, but also refers to antibody fragments, more particularly to antigen-binding fragments thereof. A fragment of an antibody comprises at least one heavy or light chain immunoglobulin domain as known in the art and binds to one or more antigen(s). Examples of antibody fragments according to the invention include a Fab, modified Fab, Fab′, modified Fab′, F(ab′)2, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). Exemplary of such antibody fragments are FabdsscFv (also referred to as BYbe®) or Fab-(dsscFv)2 (also referred to as TrYbe®, see WO2015197772 for instance). Antibody fragments as defined above are known in the art.

### DETAILED DESCRIPTION OF THE INVENTION

This disclosure relates to processes (or methods) for production of recombinant proteins. More specifically, it describes processes (or methods) to produce recombinant protein in mammalian cells and to improve cell culture performances (such as cell growth, specific productivity and/or titre/yield). In particular the invention is based on the findings from the inventors that by applying specific conditions during N−1 stage, such as controlling the feeding strategy at the N−1 stage (i.e. in the seed bioreactor) it was possible to improve cell growth (e.g. increase growth rate, increase cell viability, decrease of the possible lag phase at the start of the N stage) and increase the yield of recombinant protein production by cells during the production phase, more particularly in high seeding density (HSD) processes (see also FIG. 9). Surprisingly the inventors have found that stressing the cells (such as via an osmotic stress) during the N−1 stage (i.e. in the seed bioreactor) run in a fed-batch mode, it was possible to improve cell growth and increase the yield of recombinant protein production in the following N stage (i.e. production bioreactor), in particular in intensified processes, such as HSD processes. It was also possible to reduce or even to avoid the lag phase observed at the start of the production phase (in the N stage) for some expressing cells. As shown herein, the timing and/or duration of the stress, such as of an osmotic stress, are important factors (e.g. osmotic stress one day before the inoculation could be sufficient to improve the production performance). The inventors have called this stress an “organised stress”. Therefore, the processes herein described rely on the N−1 stage being performed in fed batch under specific conditions (such as specific stress conditions alternatively named “organised stress” conditions), i.e. the control of feeding mode and/or engineering parameters during the N−1 stage, especially when integrated to an intensified process, for the production of the recombinant protein of interest during the following N stage (i.e. production stage).

This disclosure describes in particular how to control the feeding conditions/engineering parameters during the N−1 stage in order to maximize the production bioreactor yield and maximize the cell growth. This disclosure provides specific examples of fed-batch processes, such as HSD fed-batch processes, for the seed bioreactor (N−1 stage) in which these parameters are controlled within the claimed ranges and details specific examples of possible modes of addition of feed(s) (bolus, including duration and timing, versus continuous/semi-continuous etc?).

In one embodiment, the invention provides a process for producing a recombinant protein in a production bioreactor, wherein the process comprises:
a. inoculating a N−1 bioreactor with mammalian cells comprising a gene that encodes the recombinant protein,
b. culturing the mammalian cells in the N−1 bioreactor run in a fed-batch mode, wherein the fed-batch is performed under specific conditions selected from:
   i. specific modes and duration of addition of the feed or of at least one of the feeds,
   ii. control of the total quantity of feed(s) to be added, and/or
   iii. control of at least one engineering parameter,
c. inoculating a N bioreactor at a seeding density of at least 2.00×10⁶ viable cells/ml with cells obtained from step (b);
d. culturing the cells in the N bioreactor under conditions that allow production of the recombinant protein,
e. and optionally harvesting the recombinant protein, purifying the recombinant protein and formulating the recombinant protein.

It was shown that in said process for producing a recombinant protein, the yield of recombinant protein was increased and/or the cell growth in the production bioreactor was improved. So that in an alternative embodiment, the invention provides a process for producing a recombinant protein in a production bioreactor, wherein the process comprises: a. inoculating a N−1 bioreactor with mammalian cells comprising a gene that encodes the recombinant protein, b. culturing the mammalian cells in the N−1 bioreactor run in a fed-batch mode, wherein the fed-batch is performed under specific conditions selected from: i. specific modes and duration of addition of the feed or of at least one of the feeds, ii. control of the total quantity of feed(s) to be added, and/or iii. control of at least one engineering parameter, c. inoculating a N bioreactor at a seeding density of at least 2.00×10⁶ viable cells/ml with cells obtained from step (b); d. culturing the cells in the N bioreactor under conditions that allow production of the recombinant protein, e. and optionally harvesting the recombinant protein, purifying the recombinant protein and formulating the recombinant protein, wherein the yield of recombinant protein and/or the cell growth in the production bioreactor are also improved. It is understood that the yield of recombinant protein is increased and/or the cell growth in the production bioreactor is improved compared to a process not comprising the specific conditions of step b) (i.e. wherein the cell growth is increased compared to cells not grown under an “organised stress”, during the N−1 stage).

In another embodiment, the invention provides a process for improving mammalian cell growth in a production bioreactor wherein the process comprises:
a. inoculating a N−1 bioreactor with mammalian cells comprising a gene that encodes the recombinant protein,
b. culturing the mammalian cells in the N−1 bioreactor run in a fed-batch mode under specific conditions selected from:
   i. specific modes and duration of addition of the feed or of at least one of the feeds,
   ii. control of the total quantity of feed(s) to be added, and/or
   iii. control of at least one engineering parameter,
c. inoculating a N bioreactor at a seeding density of at least 2.00×10⁶ viable cells/ml with cells obtained from step (b);
d. culturing the cells in the N bioreactor under conditions that allow production of the recombinant protein,
e. and optionally harvesting the recombinant protein, purifying the recombinant protein and formulating the recombinant protein.

The skilled person would understand that the improvement on the cell growth is observed in comparison with a process during which the cells are not grown according to the specific conditions of step b) (i.e, wherein the cell growth is increased compared to cells not grown under an “organised stress”, during the N−1 stage).

It was shown that in said process for improving mammalian cell growth in a production bioreactor, the yield of recombinant protein was also increased. So that in an alternative embodiment, the invention provides a process for improving mammalian cell growth in a production bioreactor wherein the process comprises: a. inoculating a N−1 bioreactor with mammalian cells comprising a gene that encodes the recombinant protein, b. culturing the mammalian cells in the N−1 bioreactor run in a fed-batch mode under specific conditions selected from: i. specific modes and duration of addition of the feed or of at least one of the feeds, ii. control of the total quantity of feed(s) to be added, and/or iii. control of at least one engineering parameter; c. inoculating a N bioreactor at a seeding density of at least 2.00×10⁶ viable cells/ml with cells obtained from step (b); d. culturing the cells in the N bioreactor under conditions that allow production of the recombinant protein, e. and optionally harvesting the recombinant protein, purifying the recombinant protein and formulating the recombinant protein, wherein the cell growth in the bioreactor is improved compared to cells not grown under the conditions of step b) and wherein the yield of recombinant protein is also increased.

In a further embodiment, the invention relates to a process for increasing the yield of production of a recombinant protein expressed by mammalian cells in culture in a production bioreactor, wherein the process comprises:
a. inoculating a N−1 bioreactor with mammalian cells comprising a gene that encodes the recombinant protein,
b. culturing the mammalian cells in the N−1 bioreactor run in a fed-batch mode under specific conditions selected from:
   i. specific modes and duration of addition of the feed or of at least one of the feeds,
   ii. control of the total quantity of feed(s) to be added, and/or
   iii. control of at least one engineering parameter,
c. inoculating a N bioreactor at a seeding density of at least 2.00×10⁶ viable cells/ml with cells obtained from step (b);
d. culturing the cells in the N bioreactor under conditions that allow production of the recombinant protein,
e. and optionally harvesting the recombinant protein, purifying the recombinant protein and formulating the recombinant protein.

The skilled person would understand that the improvement on the yield of production of a recombinant protein is observed in comparison with a process during which the cells are not grown according to the specific conditions of step b) (i.e, wherein the yield of production of a recombinant protein is increased compared to cells not grown under an “organised stress”, during the N−1 stage).

It was shown that in said process for increasing the yield of production of a recombinant protein, the cell growth in the bioreactor was improved. So that in an alternative embodiment, the invention relates to a process for increasing the yield of production of a recombinant protein expressed by mammalian cells in culture in a production bioreactor, wherein the process comprises: a. inoculating a N−1 bioreactor with mammalian cells comprising a gene that encodes the recombinant protein, b. culturing the mammalian cells in the N−1 bioreactor run in a fed-batch mode under specific conditions selected from: i. specific modes and duration of addition of the feed or of at least one of the feeds, ii. control of the total quantity of feed(s) to be added, and/or iii. control of at least one engineering parameter, c. inoculating a N bioreactor at a seeding density of at least 2.00×10⁶ viable cells/ml with cells obtained from step (b); d. culturing the cells in the N bioreactor under conditions that allow production of the recombinant protein, e. and optionally harvesting the recombinant protein, purifying the recombinant protein and formulating the recombinant protein, wherein the yield of production is increased compared to cells not grown under the conditions of step b) and wherein the cell growth in the bioreactor is also improved.

The skilled person would understand that in order to perform step (c) with cells obtained from step (b), a certain level of viable cell density should be obtained at the end of step (b). Therefore, also encompassed by the invention are a process for producing a recombinant protein in a production bioreactor, a process for improving mammalian cell growth in a production bioreactor and/or a process for increasing the yield of production of a recombinant protein expressed by mammalian cells in culture in a production bioreactor, wherein the process comprises:
a. inoculating a N−1 bioreactor with mammalian cells comprising a gene that encodes the recombinant protein,
b. culturing the mammalian cells in the N−1 bioreactor run in a fed-batch mode under specific conditions until at least 8×10⁶ viable cells/ml are obtained, and wherein the specific conditions are selected from:
   i. specific modes and duration of addition of the feed or of at least one of the feeds,
   ii. control of the total quantity of feed(s) to be added, and/or
   iii. control of at least one engineering parameter,
c. inoculating a N bioreactor at a seeding density of at least 2.00×10⁶ viable cells/ml with cells obtained from step (b);
d. culturing the cells in the N bioreactor under conditions that allow production of the recombinant protein,
e. and optionally harvesting the recombinant protein, purifying the recombinant protein and formulating the recombinant protein.

Although it is important, according to the invention, to run the N−1 stage in fed-batch mode, the N stage can be run according to any mode, such as perfusion, batch or fed-batch. Preferably the N stage is run in perfusion or fed-batch mode. The skilled person knows how to run production stage in perfusion, batch or fed-batch mode.

In the context of the invention as a whole, the improvement of cell growth in the production bioreactor can be for instance an increase of the cell growth, as determined by an increase of the VCC compared to cells not grown under an “organised stress” during the N−1 stage and/or a reduction of the lag phase that can happen at the start of the production phase, compared to cells not grown under an “organised stress”, during the N−1 stage. Increasing the VCC or reducing the lag phase, should there be a lag phase, will allow the production of recombinant protein not to be delayed.

In the context of the invention as a whole, the specific conditions that are applied during the fed-batch N−1 stage are considered as stress conditions for the cells. Because these conditions (alternatively called stress conditions) are controlled, they can be alternatively named “organised stress” conditions. In all of the processes herein described step b) can alternatively be described as:
b) culturing the mammalian cells in the N−1 bioreactor run in a fed-batch mode under specific stress conditions, wherein said specific stress conditions are selected from: i. specific modes and duration of addition of the feed or of at least one of the feeds, ii. control of the total quantity of feed(s) to be added, and/or iii. control of at least one engineering parameter, or yet as
b) culturing the mammalian cells in the N−1 bioreactor run in a fed-batch mode under specific organized stress conditions are applied and wherein said specific organized stress conditions are selected from: i. specific modes and duration of addition of the feed or of at least one of the feeds, ii. control of the total quantity of feed(s) to be added, and/or iii. control of at least one engineering parameter.

In the context of the invention as a whole, the mammalian cells comprising a gene that encodes the recombinant protein (in other words, the mammalian cells expressing the recombinant proteins) are preferably cultivated in a fed batch mode at least 4 days during the N−1 stage but preferably no more than 7 days, more preferably at least 5 days but not more than 7 days, such as 5 days, 6 days or 7 days. The skilled person could perform a longer N−1 stage however there are associated risks such as a decrease cell viability or start of the production that is to be avoided for a N−1 stage. The feeds (at least the main feed and/or the secondary feed if any) are preferably added daily. The feeds can be added as of day 0, day 1, day 2, day 3 or day 4 after the start if the N−1 stage (i.e. day 0 being the day of inoculation) and for a duration of at least 2 days, at least 3 days, such as 3 days, 4 days, 5 days or 6 days. Typically the last feed will be added until the day before the last day of culture. So that as a non-limiting example, if the N−1 stage has a duration of 7 days and the feeds are added as of day 3, for a duration of 4 days, the last feeds will be added on day 6. in another non-limiting example, if the N−1 stage has a duration of 6 days and the feeds are added as of day 0, for a duration of 6 days, the last feeds will be added on day 5.

In the context of the invention as a whole, one of the conditions to perform an “organised stress” is the duration of the addition of the feed or of at least one of the feeds. Preferably, such a feed of step (b) is added daily as a bolus. Preferably, i) at least the main feed of step (b) is added daily as a bolus, and/or ii) at least one of the secondary feeds of step (b) (if any) is added daily as a bolus. The daily bolus is preferably added in about 3 hours or less, in about 2 hours or less or in about 1 hour or less. If there are more than one feed to be added (such as one main feed and one secondary feed), preferably all the feeds are added daily as a bolus in step (b). In such a case, the daily boluses are preferably added in about 3 hours or less, in about 2 hours or less or in about 1 hour or less. The duration of the addition of the different feeds (when there are different feeds) do not need to be similar. In a non-limiting example, the main feed can be added daily as a bolus over 3 hours and the secondary feed(s) can be added daily as a bolus over 1 hour. In another non-limiting example, the main feed can be added daily as a bolus over 1 hour and the secondary feed(s) can be added daily as a bolus over 30 minutes. The skilled person would understand that if they wish the at least one feed can alternatively be the feed comprising the carbon source, as long as it is sufficient to provoke a stress condition.

In the context of the invention as a whole, another of the conditions to perform an “organised stress” is the total quantity of feeds added during step (b). Total quantity of feeds is important and should preferably represent at least about 3.5% of the culture start volume but preferably no more than 30% per culture start volume. Therefore, in the context of the invention as a whole, the total quantity of feeds added during step (b) preferably represent at least about 3.5% of the culture start volume but preferably no more than 30% per culture start volume. Alternatively the total quantity of feeds added during step (b) preferably represent at least about 3.5% of the culture start volume but preferably no more than 25% per culture start volume (in other words the total quantity of feeds added during step (b) preferably represent from about 3.5% to about 30% of the culture start volume), such as about 3.5, 4.0, 4.5, 5.0, 5.5, 6.0, 7.0, 8.0, 9.0, 10.0, 15.0, 20.0, 25.0 or 25.0% of the culture start volume. As a non-limiting example, should the total quantity of feeds added during step (b) represent 3.5% of the culture start volume and the culture start volume be 100 L, that means that a total of 3.5 L of feeds will be added during the N−1 stage, leading to a final culture volume of 103.5 L. As another non-limiting example, should the total quantity of feeds added during step (b) represent 4.2% of the culture start volume and the culture start volume be 500 L, that means that a total of 21 L of feeds will be added during the N−1 stage, leading to a final culture volume of 521 L. In yet another non-limiting example, should the total quantity of feeds added during step (b) represent 15% of the culture start volume and the culture start volume be 200 L, that means that a total of 30 L of feeds will be added during the N−1 stage, leading to a final culture volume of 230 L.

In the context of the invention as a whole, another of the conditions to perform an “organised stress” is the control of at least one engineering parameter during step (b). Preferably, the at least one engineering parameter that is controlled during step (b) is the specific power input. More preferably, the specific power input in step (b) is controlled daily and said specific power input in step (b) should preferably reach at least 100 W/m³ for all or part of the duration of step (b). Preferably, the specific power input in step (b) reaches at least 100 W/m³, at least 110 W/m³, at least 120 W/m³, at least 130 W/m³, at least 140 W/m³, at least 150 W/m³, at least 160 W/m³ or at least 170 W/m³ for all or part of the duration of step (b). In a non-limiting example, should the duration of step (b) be 6 days, one may control the specific power input at about 120 W/m³ for the whole duration of this step (i.e. from day 0 to day 6). In another non-limiting example, should the duration of step (b) be 7 days, one may control the specific power input at about 140 W/m³ for only 5 days (i.e. part of the duration of step (b)) e.g. from day 1 to day 5.

It was shown by the inventors that it was mainly (alternatively, it was only) the power input (P/V) applied in the seed bioreactor, rather than the P/V applied in the production bioreactor, which had a significant impact on the cell culture production. Alternatively, it seems that the difference (or the ratio) between the P/V in seed bioreactor and production bioreactor remains a potential factor significantly impacting cell culture production performance. In other words, the difference between the mechanical stress brought by the power input in the seed and production bioreactors remain a potential factor impacting cell culture production performance: the conditions with high P/V in the seed bioreactor were associated to a lower P/V (lower mechanical stress) in the production step. As shown in the example section, the conditions leading to the best cell culture performances during production stage were those with high P/V in the seed bioreactor associated to a lower P/V in the production step. Therefore, in an alternative, not only the at least one engineering parameter that is controlled during step (b) is the specific power input but there is an additional engineering parameter that is controlled during step (d), i.e. the specific power input in the production bioreactor, so that a certain ratio between the power input in the seed bioreactor (P/V SBR) and the power input in the production bioreactor (P/V PBR) is respected. Preferably, the ratio P/V SBR:P/V PBR is at least 1.2:1, at least 1.25:1, at least 1.30:1, at least 1.35:1, at least 1.40:1, at least 1.45:1, at least 1.50:1, at least 1.55:1, at least 1.60:1, at last 1.65:1, at least 1.70:1, at least 1.80:1, at least 1.90:1 or yet at least 2:1. The best ratio can be determined during initial experiments. Therefore, the processes according to the invention as a whole can further comprise a preliminary step of performing at least one set of initial experiments to determine the ratio P/V SBR:P/V PBR leading to an improved mammalian cell growth in a production bioreactor and/or an improved yield of production of a recombinant protein expressed by mammalian cells in culture in a production bioreactor compared to a standard process. It has to be understood that the at least one set of initial experiments does not need to be repeated each time the processes according to the invention are performed. In other words, once the conditions are determined, in at least one set of initial experiments, for one specific clone, under given conditions, there is no need to control it each time the processes according to the invention are to be performed.

Therefore, also encompassed by the invention are a process for producing a recombinant protein in a production bioreactor, a process for improving mammalian cell growth in a production bioreactor and/or a process for the yield of production of a recombinant protein expressed by mammalian cells in culture in a production bioreactor, wherein the process comprises:
0. optionally performing at least one set of initial experiments to determine the ratio P/V SBR:P/V PBR to be preferably maintained at the production stage,
a. inoculating a N−1 bioreactor with mammalian cells comprising a gene that encodes the recombinant protein,
b. culturing the mammalian cells in the N−1 bioreactor run in a fed-batch mode under “organised stress” conditions until at least 8×10⁶ viable cells/ml are obtained, and wherein the “organised stress” conditions are selected from:
   i. specific modes and duration of addition of the feed or of at least one of the feeds,
   ii. the control of the total quantity of feed(s) to be added, and/or
   iii. the control of at least one engineering parameter, wherein said at least one engineering parameter is the specific power input,
c. inoculating a N bioreactor at a seeding density of at least 2.00×10⁶ viable cells/ml with cells obtained from step (b);
d. culturing the cells in the N bioreactor under conditions that allow production of the recombinant protein, including the step of controlling at least one engineering parameter, wherein said at least one engineering parameter is the specific power input so that the ratio P/V SBR:P/V PBR determined in the optional step (0) or in previous initial experiments is maintained,
e. and optionally harvesting the recombinant protein, purifying the recombinant protein and formulating the recombinant protein.

Although a combination of the three conditions to reach an organised stress leads to the best results for producing a recombinant protein in a production bioreactor, improving mammalian cell growth in a production bioreactor and/or the yield of production of a recombinant protein expressed by mammalian cells in culture in a production bioreactor, one of them is already enough to lead to acceptable results and may be sufficient depending on the VCC obtained at the end of the N−1 stage. Performing one, two or three of the “organised stress” conditions will depend on the producing cells. The best condition(s) can be determined during initial experiments. Therefore, the processes according to the invention as a whole can further comprise a preliminary step of performing at least one initial experiment to determine the VCC at the end of the N−1 stage and to determine which conditions to apply during the N−1 stage to reach preferably at least 8×10⁶ viable cells/ml with cells obtained from step (b) as well as to be able to produce a recombinant protein in a production bioreactor, improve mammalian cell growth in a production bioreactor and/or the yield of production of a recombinant protein expressed by mammalian cells in culture in a production bioreactor compared to a standard process. It has to be understood that this initial experiment does not need to be repeated each time the processes according to the invention are performed. In other words, once the conditions are determined, in at least one initial experiment, for one specific clone, under given conditions, there is no need to control it each time the processes according to the invention are to be performed.

Therefore, also encompassed by the invention are a process for producing a recombinant protein in a production bioreactor, a process for improving mammalian cell growth in a production bioreactor and/or a process for the yield of production of a recombinant protein expressed by mammalian cells in culture in a production bioreactor, wherein the process comprises:
0. optionally performing at least one initial experiment to determine the VCC at the end of the N−1 stage and/or optionally performing at least one initial experiment to determine the duration of the feeds during step (b), the total quantity of feeds to be added during step (b) and/or the specific power input to be used during step (b)
a. inoculating a N−1 bioreactor with mammalian cells comprising a gene that encodes the recombinant protein,
b. culturing the mammalian cells in the N−1 bioreactor run in a fed-batch mode under “organised stress” conditions until at least 8×10⁶ viable cells/ml are obtained, and wherein the “organised stress” conditions are selected from:
   i. specific modes and duration of addition of the feed or of at least one of the feeds,
   ii. the control of the total quantity of feed(s) to be added, and/or
   iii. the control of at least one engineering parameter,
c. inoculating a N bioreactor at a seeding density of at least 2.00×10⁶ viable cells/ml with cells obtained from step (b);
d. culturing the cells in the N bioreactor under conditions that allow production of the recombinant protein,
e. and optionally harvesting the recombinant protein, purifying the recombinant protein and formulating the recombinant protein.

Although the inoculation of the N bioreactor could be performed with lower seeding density (e.g. below 2.00×10⁶ viable cells/ml, below 1.50×10⁶ viable cells/ml, below 1.00×10⁶ viable cells/ml or even below 0.50×10⁶ viable cells/ml), the results in term of production of recombinant protein, increase in cell growth and/or yield of production of a recombinant protein would be better with density of at least about 2.00×10⁶ viable cells/ml (i.e. high seeding density). Therefore, in the context of the invention as a whole, the inoculation of the N bioreactor (step c)) is preferably performed at a seeding density of at least about 2.00×10⁶ viable cells/ml with cells obtained from the N−1 bioreactor. Alternatively, the N bioreactor of step (c) is inoculated at a seeding density at least 3.00×10⁶ viable cells/ml, at least 4.00×10⁶ viable cells/ml, at least 5.00×10⁶ viable cells/ml, at least 6.00×10⁶ viable cells/ml, at least 7.00×10⁶ viable cells/ml, at least 8.00×10⁶ viable cells/ml, at least 9.00×10⁶ viable cells/ml, or at least 10.00×10⁶ viable cells/ml. The skilled person would understand that “10.00×10⁶ viable cells/ml” is not a limit and higher seeding density could be foreseen, depending on the yield of the N−1 bioreactor, with regard to the cell density obtained at the end of step (b).

In order to be able to inoculate the N bioreactor under a high seeding density strategy, it is preferably to reach a (viable) cell density of at least about 8.00×10⁶ viable cells/ml at the end of the N−1 stage (end of step (b)). It is also preferable not to reach too high cell density at the end of this stage as this would likely mean that the cells were cultivated for a too long period of time, possibly impact their quality and their productivity during the N stage. Therefore, in the context of the invention as a whole, the (viable) cell density obtained at the end of the N−1 stage (end of step b)) is preferably at least about 8.00×10⁶ viable cells/ml and up to about 30.00×10⁶ viable cells/ml. Alternatively, the (viable) cell density obtained at the end of the N−1 stage (end of step b)) is preferably at least 9.00×10⁶ viable cells/ml, at least 10.00×10⁶ viable cells/ml, at least 15.00×10⁶ viable cells/ml, at least 20.00×10⁶ viable cells/ml, at least 25.00×10⁶ viable cells/ml but preferably not more than 30.00×10⁶ viable cells/ml.

In the context of the invention as a whole, the culture medium at the start of the culture (alternatively herein named basal medium; medium used for both the N−1 stage—medium for step (a)—and N stage—medium for step (c)) is preferably a protein- and serum-free culture medium. Said protein- and serum-free culture medium can be a commercially available medium or a (homemade or commercially available) chemically defined medium. Such medium can thus be used at the start of the N−1 stage (initial medium for step (a)) and at the start of the N stage (initial medium for step (c)).

In the context of the invention as a whole, the main feed medium can be any main feed medium. In an example, this main feed medium does not comprise Cys (neither cysteine nor cystine), Trp and Tyr and these components are brought via at least one additional feed (such additional feed will be the secondary feed or one of the secondary feeds).

In the context of the invention as a whole, the process is preferably carried out at large scale, such as in a bioreactor preferably with a volume of equal or more than 50 L, equal or more than 100 L, equal or more than 500 L, equal or more than 1000 L, equal or more than 2,000 L, equal or more than 55,000 L, equal or more than 10,000 L or equal or more than 20,000 L. In other words, the mammalian cells producing the recombinant proteins are cultivated in a bioreactor (such as a production bioreactor), preferably with a volume of equal or more than 50 L, equal or more than 100 L, equal or more than 500 L, equal or more than 1000 L, equal or more than 2,000 L, equal or more than 5,000 L, equal or more than 10,000 L or equal or more than 20,000 L.

In the context of the invention as a whole, suitable mammalian host cells (also named mammalian cells) include Chinese Hamster Ovary (CHO cells), lymphocytic cell lines, e.g., NSO myeloma cells and SP2 cells, COS cells, myeloma or hybridoma cells. In a preferred embodiment, the mammalian cell is a CHO cell. Suitable types of CHO cells may include CHO-K1, CHOK1-SV, dhfr-CHO, such as CHO-DG44, CHO-DXB11, CHO-DXB1, or yet CHO-S cells. The host cells are preferably stably transformed or transfected with expression vectors encoding the recombinant protein of interest. In the context of the invention as a whole, the recombinant protein is a protein such as a cytokine, a growth factor, a hormone, a fusion protein or an antibody. Should the protein be an antibody, it can be for instance a chimeric antibody, a humanised antibody or a fully human antibody and is preferably IgGs such as IgG1, IgG2, IgG3 or IgG4. Alternatively, it can be any kind of proteins as per the definition herein given.

The processes according to the invention can further comprise the step of recovering the cell culture fluid (CCF) comprising the recombinant protein (harvest step), in other words the step of harvesting the recombinant protein. Subsequently to the harvest, the recombinant protein may be purified, e.g. if the protein is an antibody, using Protein A chromatography and other chromatographic/filtration steps. The processes further optionally comprise a step of formulating the purified recombinant protein, e.g. into a formulation with a high protein concentration, such as a concentration of 10 mg/ml or more, e.g. 50 mg/ml or more, such as 100 mg/ml or more, 150 mg/ml or more or yet 200 mg/mL or more. Without any limitation, the formulation can be a liquid formulation, lyophilised formulation or a spray-dried formulation.

### DESCRIPTION OF THE FIGURE

FIG. 1: A) Cell growth in N−1 bioreactor for cells expressing mAb1. B) Cumulative IVCC in N bioreactor for cells expressing mAb1.

FIG. 2: Summary of multivariate linear regression fit for cumulative IVCC in N bioreactor.

FIG. 3: A) Cell growth profiles in N bioreactor for cells expressing mAb1. B) Titres in N bioreactor for cells expressing mAb1.

FIG. 4: N bioreactor's data for cells expressing mAb1. A) Cell viability; B) Viable cell count. C) Titres (normalised). D) Legend for FIGS. 4A-4C.

FIG. 5: A) Cell growth in N−1 bioreactor for cells expressing mAb1. B) Cumulative IVCC in N bioreactor for cells expressing mAb1.

FIG. 6: A) Cell growth profiles for cell expressing mAb1. B) Titres in the N bioreactor for cell expressing mAb1.

FIG. 7: A) Cell growth profiles in N bioreactor for cell expressing mAb2. B) Titres in N bioreactor for cell expressing mAb2.

FIG. 8: A) Cell growth profiles in N bioreactor for cell expressing mAb2. B) Titres in the N bioreactor for cell expressing mAb2.

FIG. 9: Design for extended lag phase scale-down model screening in a stirred glass vessel bioreactor from seed bioreactor (N−1) to production bioreactor (N). “Feed 1” corresponds to the main feed and “Feed 2” corresponds to the secondary feed.

FIG. 10: Impact of specific power input in seed bioreactor on cell culture performance of intensified processes for cells expressing mAb2. The “organized stress” concept was applied to two 2000 L cell culture production bioreactors in order to verify the small-scale data. The specific power input in the seed bioreactor (400 L bioreactor) was varied (Table 3). The viable cell density, cell viability, specific production rates of glucose and lactate profiles in the intensified production bioreactors are presented.

### EXAMPLES

### Cell Line, Cell Culture and Experimental Procedure

Two different production CHO-DG44 cell lines were used, respectively producing: mAb-1 (a full IgG4 antibody having a pl of 5.7-5.9) and mAb-2 (a trispecific antibody having a pl of 8.9-9.2).

Two set of media were used:
First source (Media from Cytiva): ActiPro™ as basal medium, Cell Boost™ 7a as a main feed (herein alternatively named CB7a) and Cell Boost™ 7b as a secondary feed bringing additional elements to those from the main feed (herein alternatively named CB7b).
Second source (modified CHO Pro media): “BM1” as basal medium, “FM 1” as a main feed and “FM 2” as a secondary feed bringing additional elements to those from the main feed

The cells were cultivated in 2 L stirred tank glass bioreactor (STR) with supply towers (C-DCUII, Sartorius Stedim Biotech) controlled by a multi-fermentation control system (MFCS, Sartorius Stedim Biotech). The bioreactors were equipped with a 3-segment blade impeller. The cultivation start volume was adapted to ensure the cultivation end volume is optimal. The production bioreactors were seeded at target seeding density (TSD) in a basal medium. The pH control of the production STR was set to 7.0 with a dead band of +0.2. The pO2 target was set to 40% air saturation. To control pO2, air, nitrogen and oxygen were sparged into the culture vessel based on a cascade controller using a predefined mixture profile. The temperature was controlled at about 36.8° C. The seed bioreactor (N−1 stage) was operated in fed-batch mode for 5 or 6 days (via addition of two different feeds). The production (N stage) was then operated in fed-experiment mode for 14 days (via addition of two different feeds). During this phase, the monoclonal antibody (mAb) was secreted into the medium. Samples were drawn daily to determine VCD, viability, off-line pH, pCO2, osmolality, glucose-lactate concentration, amino acid concentration and mAb concentration (stocked at −80° C.). Antifoam was added manually on demand every day to control the build-up of foam. 72 hours after inoculation, continuous nutrient feeding was started with a predetermined rate. A glucose bolus feed was added to the culture when the glucose concentration dropped below a given threshold. Glucose concentrations were measured daily. Samples for the amino acid analysis were taken before the addition of the feeds. The extracellular concentrations after feeding were computed based on the feed composition and measured nutrients concentration before feed addition. At the end of the production phase, after harvest, the cell culture supernatant samples (i.e. harvest cell culture fluids) were purified with a Protein A purification on TECAN automated system.

### Analytical Methods

Cell were counted by using a VI-CELL® XR (Beckman-Coulter, Inc.) automated cell counting device that operated based on trypan blue exclusion. Glucose and lactate levels in the culture medium were determined using a NOVA 400 BioProfile automated analyzer (Nova Biomedical) or a Cedex Bio HT (Roche). A model 2020 freezing point osmometer (Advanced Instruments, Inc.) was used for osmolality determination. Offline gas and pH measurements were performed with a model BioProfile pHOx® blood gas analyzer (Nova Biomedical Corp.). Metabolites concentrations were determined daily using a CedexBioHT system (Roche). Product titre analysis were performed with protein A high-pressure liquid chromatography (HPLC) with cell culture supernatant samples which were stored at −80° C. prior to analysis. The relative percentage of main, acidic (APG for Acidic Peak Group) and basic (BPG for Basic Peak Group) isoform of the purified mAb is determined by Imaged Capillary Electrophoresis (ProteinSimple ICE3). Aggregate (HMWS), monomer and fragment (LMWS) levels of the purified mAb are determined by size exclusion chromatography (SE-UPLC) or protein A HPLC gradient.

### Example 1—High Feeding Regime, Mode of Addition of the Feeds and Specific Power Input of Seed Bioreactor Impact Cell Culture Performance in Production Bioreactor (mAb1)

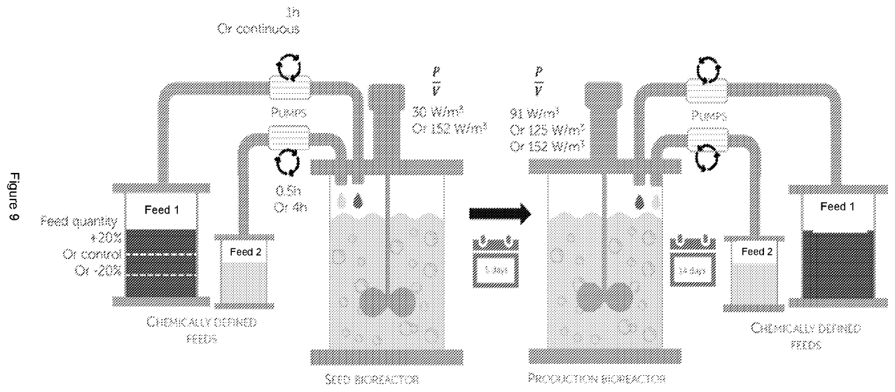

For this experiment, 5 N−1 2 L seed bioreactors (SBR) were inoculated with CHO cells producing mAb-1 at a seeding density of 0.35×10⁶ cells/mL and various conditions of feeding mode, daily feed addition duration and specific power inputs were tested (see Table 1) for 5 days. Fifteen production bioreactors (PBR) (see Table 1 as well as FIG. 9) were then inoculated at 3.0×10⁶ cells/mL from the different N−1 seed bioreactors described above. The objective was to assess the impact of various parameters in seed bioreactor, i.e. feed quantity added, mode of addition (bolus or continuous) and specific power inputs, on production bioreactor cell growth. The specific power input was also varied in the production bioreactors for some conditions.

TABLE 1
Experimental conditions of example 1

| | | CB7a per | | | Daily | | |
|---|---|---|---|---|---|---|---|
| | | culture | | Daily | Feed | Specific | Specific |
| | | start | Feeding | Feed | CB7b | power | power |
| Production | Quantity | volume | mode for | CB7a | duration | input | input |
| Bioreactor | of Feeds | (%) in | CB7a in | duration | (bolus) | (W/m³) | in PBR |
| ID | in SBR | SBR | SBR | in SBR | in SBR | in SBR | (W/m³) |
| 1 | Control | ≈3.50 | Bolus | 1 h | 30 min | 30 | 91 |
| | mAb-1 | | | | | | |
| 2 | Control | ≈3.50 | Bolus | 1 h | 30 min | 30 | 91 |
| | mAb-1 | | | | | | |
| 3 | Control | ≈4.20 | Bolus | 1 h | 30 min | 152 | 125 |
| | mAb-1 +20% | | | | | | |
| | CB7a | | | | | | |
| 4 | Control | ≈2.80 | Bolus | 1 h | 30 min | 152 | 125 |
| | mAb-1 −20% | | | | | | |
| | CB7a | | | | | | |
| 5 | Control | ≈3.50 | Bolus | 1 h | 30 min | 152 | 91 |
| | mAb-1 | | | | | | |
| 6 | Control | ≈3.50 | Bolus | 1 h | 30 min | 152 | 91 |
| | mAb-1 | | | | | | |
| 7 | Control | ≈3.50 | Bolus | 1 h | 30 min | 30 | 125 |
| | mAb-1 | | | | | | |
| 8 | Control | ≈3.50 | Bolus | 1 h | 30 min | 30 | 125 |
| | mAb-1 | | | | | | |
| 9 | Control | ≈3.50 | Bolus | 1 h | 30 min | 152 | 152 |
| | mAb-1 | | | | | | |
| 10 | Control | ≈3.50 | Bolus | 1 h | 30 min | 152 | 125 |
| | mAb-1 | | | | | | |
| 11 | Control | ≈3.50 | Bolus | 1 h | 30 min | 152 | 125 |
| | mAb-1 | | | | | | |
| 12 | Control | ≈3.50 | Continuous | 24 h | 30 min | 152 | 125 |
| | mAb-1 | | | | | | |
| 13 | Control | ≈3.50 | Continuous | 24 h | 30 min | 152 | 125 |
| | mAb-1 | | | | | | |
| 14 | Control | ≈2.80 | Bolus | 1 h | 30 min | 152 | 125 |
| | mAb-1 −20% | | | | | | |
| | CB7a | | | | | | |
| 15 | Control | ≈4.20 | Bolus | 1 h | 30 min | 152 | 125 |
| | mAb-1 +20% | | | | | | |
| | CB7a | | | | | | |

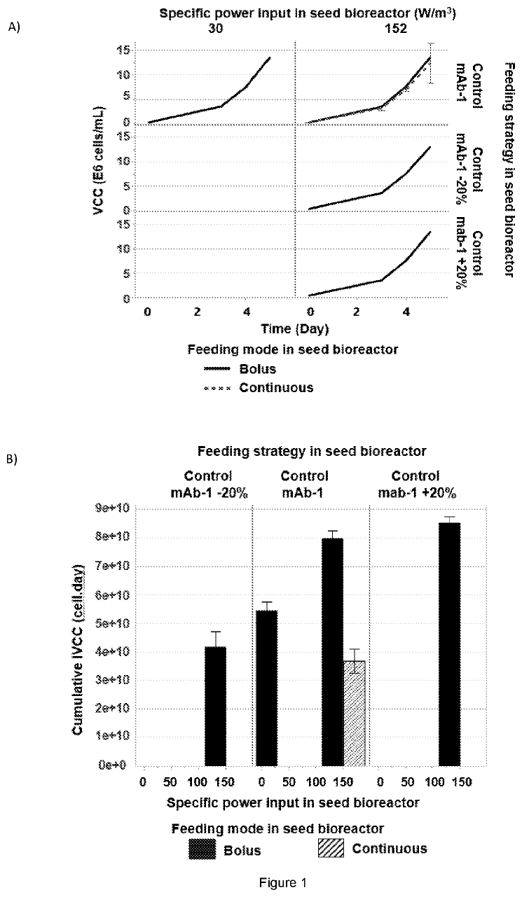

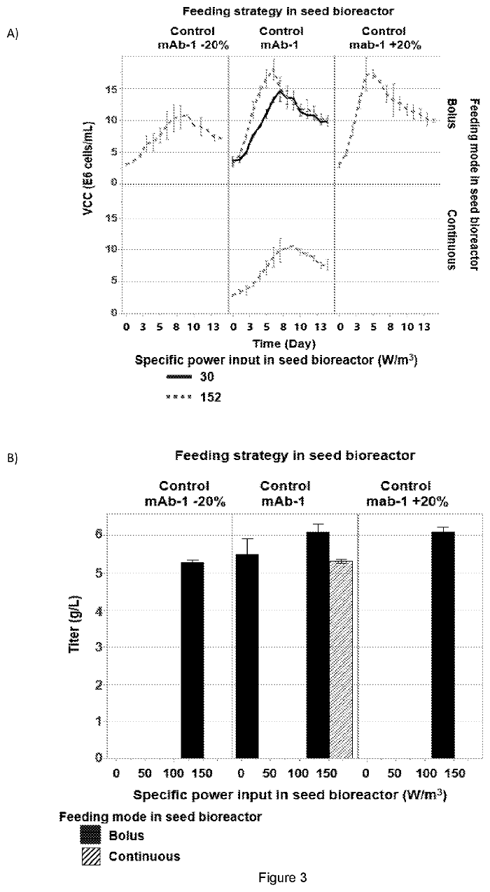

Comparable cell growths were observed during the N−1 stage, with a maximum VCC around 14×10⁶ cells/mL, for all of the tested conditions (FIG. 1A). The results shown in FIG. 1B (i.e. cumulative IVCC) and FIG. 3A (VCC) highlight that the cell growth in production bioreactors (N stage) was lower for the conditions with:
lower feed quantity added in seed bioreactor (N−1 stage),
lower specific power inputs in the seed bioreactor and/or continuous mode of addition of the main feed in the seed bioreactor.

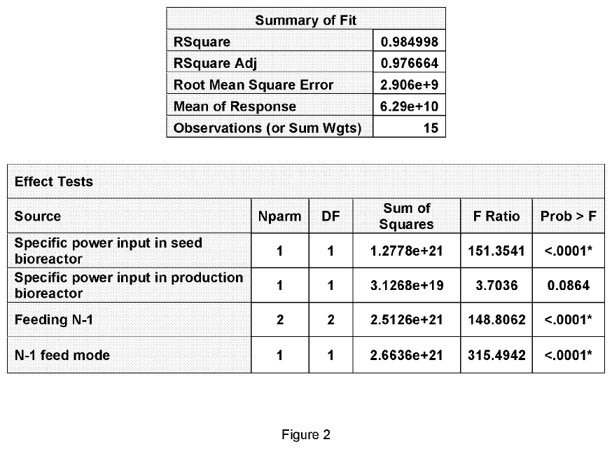

To the contrary, as shown in FIG. 2, the specific power input in the subsequent production bioreactors had no significant impact on the cell growth in production bioreactors.

The effects on titre were similar to those on cell growth, i.e. the titre was lower for the conditions with:
lower feed quantity added in seed bioreactor (N−1 stage),
lower specific power inputs in the seed bioreactor and/or
continuous mode of addition of the main feed in the seed bioreactor (see FIG. 3B)

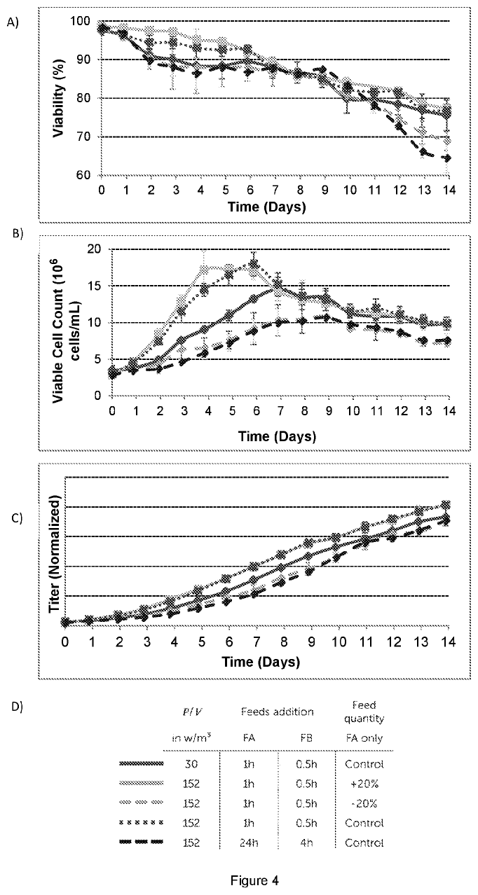

This example shows that by culturing the mammalian cells in the N−1 bioreactor run in a fed-batch mode under specific conditions selected from i) specific modes and duration of addition of at least one of the feeds, ii) control of the total quantity of feed(s) to be added, and/or iii) control of at least one engineering parameter, such as the specific power input, higher cell growth and increased final productivity can be obtained in the N bioreactor (see FIGS. 4A-4C). In other words, by controlling the mode and duration of feeding, the total quantity of feed added and/or the specific power input during the N−1 stage, it was possible to improve mammalian cell growth and increase the yield of production of a recombinant protein expressed by mammalian cells in the N bioreactor. With regard to the specific power input (P/V), in view of the results in this example, it appeared that the P/V applied in the seed bioreactor had a significant impact on the cell culture production. More specifically, the difference between the P/V in seed bioreactor and production bioreactor (higher P/V in the SBR compared to the P/V in the PBR) remains a potential factor impacting cell culture production performance. Although not limiting, the results showed that a ratio P/V (SBR):P/V PBR of at least 1.20:1 led to higher cell culture performance.

### Example 2—Mode of Addition of the Feeds in Seed Bioreactor Impacts Cell Culture Performance in Production Bioreactor (mAb1)

For this experiment, 4×10 L N−1 stage bioreactors (SBR) were inoculated with CHO cells producing mAb1 at 0.35×10⁶ cells/mL under various conditions (see Table 2) for 6 days. 8×2 L production bioreactors (PBR) have then been inoculated from those N−1 bioreactors at a seeding density of 3.0×10⁶ cells/mL in fed-batch process as described in the materials and methods. In this experiment, multiple conditions with duration of addition of daily feeds of Cell Boost™ 7a and cell Boost™ 7b were assessed (see Table 2).

TABLE 2
Experimental conditions in the seed bioreactor (N-1 stage) of example 2

| | | | | Daily | Daily | | Specific | Specific |
|---|---|---|---|---|---|---|---|---|
| | Feeding | CB7a per | | Feed | Feed | | power | power |
| Production | Strategy | culture start | Feeding | CB7a | CB7b | | input | input |
| Bioreactor | (quantity) | volume (%) | mode in | duration | duration | | (W/m³) | in PBR |
| ID | in SBR | in SBR | SBR | in SBR | in SBR | | in SBR | (W/m³) |
| 16 | Control | ≈3.50 | Bolus | 1 h | 30 | min | 159 | 125 |
| | mAb-1 | | | | | | | |
| 17 | Control | ≈3.50 | Continuous | 24 h | 4 | h | 159 | 125 |
| | mAb-1 | | | | | | | |
| 18 | Control | ≈3.50 | Bolus | 1 h | 30 | min | 159 | 125 |
| | mAb-1 | | | | | | | |
| 19 | Control | ≈3.50 | Continuous | 24 h | 30 | min | 159 | 125 |
| | mAb-1 | | | | | | | |
| 20 | Control | ≈3.50 | Bolus | 1 h | 30 | min | 159 | 125 |
| | mAb-1 | | | | | | | |
| 21 | Control | ≈3.50 | Continuous | 24 h | 4 | hours | 159 | 125 |
| | mAb-1 | | | | | | | |
| 22 | Control | ≈3.50 | Bolus | 1 h | 30 | min | 159 | 125 |
| | mAb-1 | | | | | | | |
| 23 | Control | ≈3.50 | Continuous | 24 h | 30 | min | 159 | 125 |
| | mAb-1 | | | | | | | |

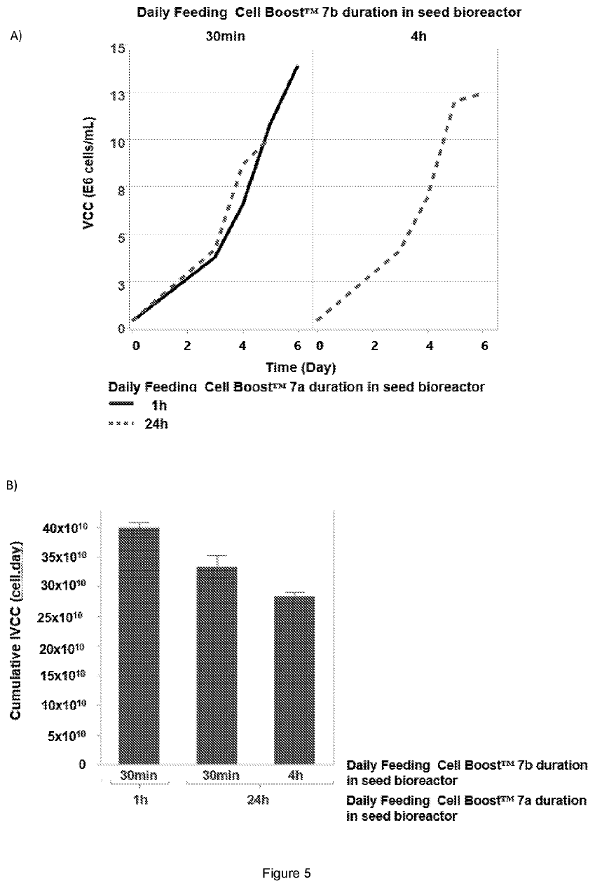

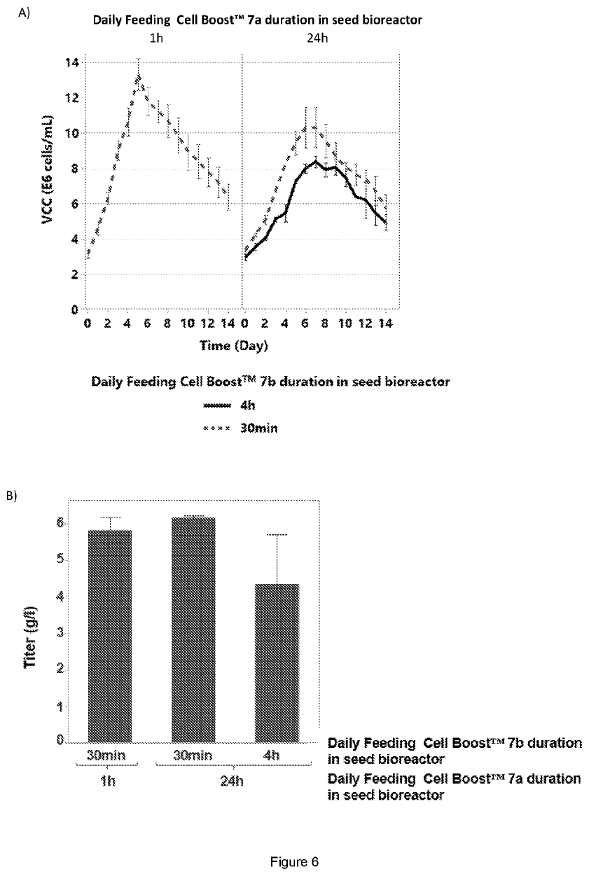

In FIG. 5A we can observe comparable cell growth profiles in the seed bioreactors for the different conditions tested, whereas FIG. 6A shows a lower cell growth in production bioreactors for the conditions with longer duration of feeds addition in seed bioreactor. The cumulative IVCC in N bioreactor is depicted in FIG. 5B. The results show a lower cell growth in production bioreactors for the conditions with longer duration of feed addition in seed bioreactor. We can observe that the impact of CB7a and CB7b are cumulative. The conditions with longer feed addition duration of both cell boost 7a and 7b show a lower final titre at the end of the 14 days production (FIG. 6B):

This example confirms the results obtained in Example 1, i.e. that by controlling the mode and duration of feeding of at least one of the feeds during the N−1 stage, it was possible to improve mammalian cell growth and increase the yield of production of a recombinant protein expressed by mammalian cells in the N bioreactor.

### Example 3? Mode of Addition of the Feeds in the Seed Bioreactor Impact Cell Culture Performance in Production Bioreactor (mAb2)

For this experiment, 4×10 L N−1 stage bioreactors (SBR) were inoculated with CHO cells producing mAb2 at 0.35×10⁶ cells/mL under various conditions (see Table 3) for 6 days. 13×2 L production bioreactors (PBR) have been inoculated with CHO cells producing mAb2 at a seeding density of 3.75×10⁶ cells/mL in fed-batch process as described in the materials and methods. In this experiment, multiple conditions with various mode of addition of FM1 were tested in seed bioreactors.

TABLE 3
Experimental conditions in the seed bioreactor (N-1 stage) of example 3

| | | | | Daily | Daily | Specific | Specific |
|---|---|---|---|---|---|---|---|
| | Feeding | CB7a per | | Feed | Feed | power | power |
| Production | Strategy | culture start | Feeding | FM1 | FM2 | input | input |
| Bioreactor | (quantity) | volume (%) | mode in | duration | duration | (W/m³) | in PBR |
| ID | in SBR | in SBR | SBR | in SBR | in SBR | in SBR | (W/m³) |
| 24 | Control | ≈4.20 | Bolus | 3 hours | 30 min | 159 | 125 |
| | mAb-2 | | | | | | |
| 25 | Control | ≈4.20 | Bolus | 3 hours | 30 min | 159 | 125 |
| | mAb-2 | | | | | | |
| 26 | Control | ≈4.20 | Continuous | 24 hours | 30 min | 159 | 125 |
| | mAb-2 | | | | | | |
| 27 | Control | ≈4.20 | Bolus | 3 hours | 30 min | 159 | 125 |
| | mAb-2 | | | | | | |
| 28 | Control | ≈4.20 | Bolus | 3 hours | 30 min | 159 | 125 |
| | mAb-2 | | | | | | |
| 29 | Control | ≈4.20 | Bolus | 3 hours | 30 min | 159 | 125 |
| | mAb-2 | | | | | | |
| 30 | Control | ≈4.20 | Continuous | 24 hours | 30 min | 159 | 125 |
| | mAb-2 | | | | | | |
| 31 | Control | ≈4.20 | Bolus | 3 hours | 30 min | 159 | 125 |
| | mAb-2 | | | | | | |
| 32 | Control | ≈4.20 | Continuous | 24 hours | 30 min | 159 | 125 |
| | mAb-2 | | | | | | |
| 33 | Control | ≈4.20 | Bolus | 3 hours | 30 min | 159 | 125 |
| | mAb-2 | | | | | | |
| 34 | Control | ≈4.20 | Continuous | 24 hours | 30 min | 159 | 125 |
| | mAb-2 | | | | | | |
| 35 | Control | ≈4.20 | Bolus | 3 hours | 30 min | 159 | 125 |
| | mAb-2 | | | | | | |
| 36 | Control | ≈4.20 | Continuous | 24 hours | 30 min | 159 | 125 |
| | mAb-2 | | | | | | |

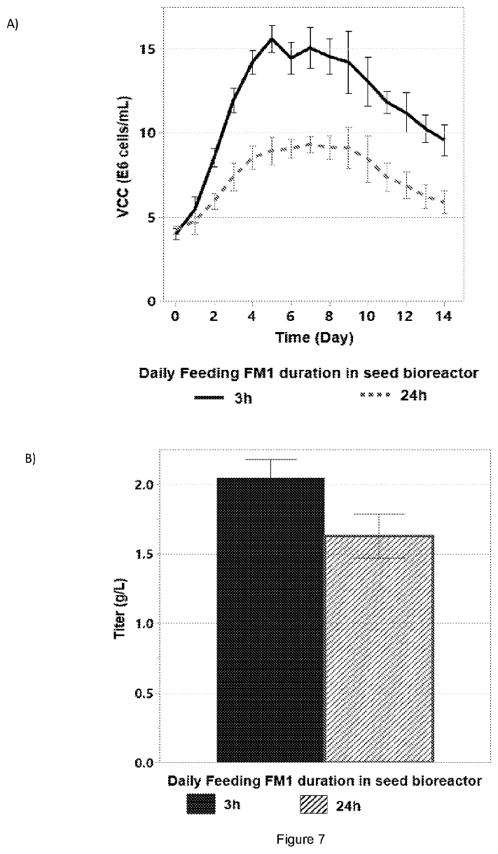

The cell growth profile is depicted in FIG. 7. The results shown in FIG. 7A show a lower cell growth in production bioreactors for the conditions with longer duration of feed addition in seed bioreactor. The final product titre at the end of 14 days productions (FIG. 7B) show a lower final productivity in production bioreactors for the conditions with longer duration of feed addition in seed bioreactor. It can be observed that short duration of addition of FM1 in seed bioreactor impact significantly the cell growth and final productivity in production bioreactor. This confirm that the mode and duration of addition of the feed in seed bioreactor (preferable in ≤3 hours) have a significant positive impact on increasing cell growth and product titre in production bioreactor, confirming the results from Examples 1 and 2 but with another cell clone (producing a different antibody) and with another media platform.

### Example 4—Quantity of Feeds Added in Seed Bioreactor Impacts Cell Culture Performance in Production Bioreactor (mAb2)

For this experiment, 4×10 L seed bioreactors (SBR) were inoculated with CHO cells producing mAb2 at 0.35×10⁶ cells/mL under various conditions (see Table 4) for 6 days. 4×2 L production bioreactors (PBR) have been inoculated with CHO cells producing mAb2 at a seeding density of 3.75×10⁶ cells/mL in fed-batch process as described in the materials and methods. In this experiment, multiple conditions with various quantity of feed added in seed bioreactor were assessed. Moreover, the specific power input in seed bioreactor has been set to a low value to be able to assess if high quantity of feed in seed bioreactor could balance the fact that the specific power input was reduced. For the same purpose, the feed was added in continuous mode in seed bioreactor.

TABLE 4
Experimental conditions in the seed bioreactor (N-1 stage) of example 4

| | | | | | | Specific | Specific |
|---|---|---|---|---|---|---|---|
| | Feeding | CB7a per | | | | power | power |
| Production | Strategy | culture start | Feeding | Daily Feed | Daily Feed | input | input |
| Bioreactor | (quantity) | volume (%) | mode in | FM1 duration | FM2duration | (W/m³) | in PBR |
| ID | in SBR | in SBR | SBR | in SBR | in SBR | in SBR | (W/m³) |
| 37 | Control | ≈4.20 | Continuous | 24 hours | 30 min | 54 | 125 |
| | mAb-2 | | | | | | |
| 38 | Control | ≈5.85 | Continuous | 24 hours | 30 min | 54 | 125 |
| | mAb-2 +40% | | | | | | |
| 39 | Control | ≈4.20 | Continuous | 24 hours | 30 min | 54 | 125 |
| | mAb-2 | | | | | | |
| 40 | Control | ≈5.85 | Continuous | 24 hours | 30 min | 54 | 125 |
| | mAb-2 +40% | | | | | | |

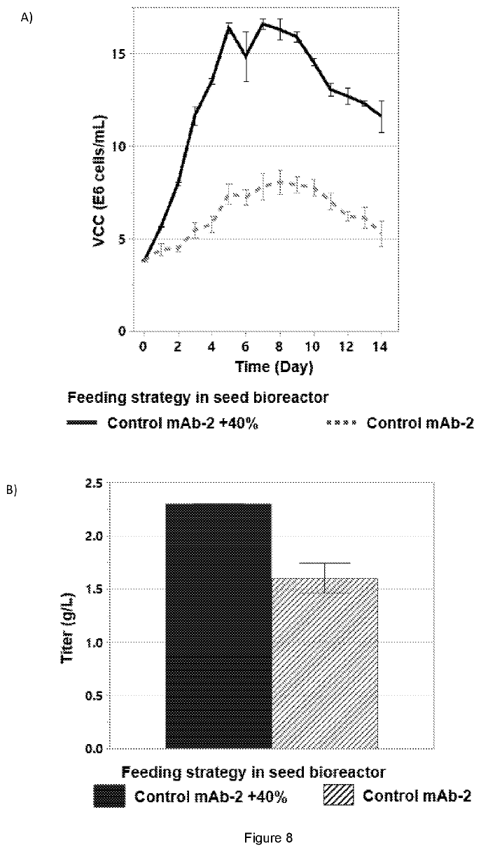

The cell growth profiles (in the production bioreactor) are depicted in FIG. 8A and show that a higher feeding quantity added in seed bioreactor compared to control condition (about 5.85% versus about 4.20% total quantity of feed added per cell culture start volume in seed bioreactor w/w) leads to increase cell growth in production bioreactor even if the specific power input in the SBR is below 90 W/m³ and FM1 is added in continuous mode in the seed bioreactor. Moreover, the final product titre at the end of 14 days of production (FIG. 8B) shows a higher final productivity in production bioreactors for the conditions with higher feeding quantity added in seed bioreactor compared to control condition (5.85% versus 4.20%). This confirms the conclusion made in the previous examples that controlling the total quantity of feed per culture start volume of seed bioreactor (such as to be higher than about 3.50%) leads to higher cell growth and increased final productivity.

### Example 5? Engineer Parameter Impacts Cell Culture Performance in Production Bioreactor (mAb1)

As shown in Example 1, the difference between the P/V in the seed and production bioreactors is a potential factor impacting cell culture production performance: the conditions with high P/V in the seed bioreactor associated to a lower P/V in the production step led to better cell culture performances.

In order to verify this finding, one 400 L seed bioreactor (SBR) was inoculated with CHO cells producing mAb1 at 0.35×10⁶ cells/mL under two conditions (see Table 5) for 6 days. One 2000 L production bioreactor (PBR) had then been inoculated with the cells at a seeding density of 3.0×10⁶ cells/mL in fed-batch process as described in the materials and methods. In this experiment, two conditions were tested (see Table 5),

TABLE 5
Experimental conditions in the bioreactors of example 5

| | | | Daily | Daily | Specific | Specific |
|---|---|---|---|---|---|---|
| | Feeding | | Feed | Feed | power | power |
| Production | Strategy | Feeding | FM1 | FM2 | input | input |
| Bioreactor | (quantity) | mode in | duration | duration | in SBR | in PBR |
| ID | in SBR | SBR | in SBR | in SBR | (W/m³) | (W/m³) |
| 41 | Control | Bolus | 1 h | 30 min | 32.8 | 81 |
| | mAb-1 | | | | | |
| 42 | Control | Bolus | 1 h | 30 min | 134 | 81 |
| | mAb-1 | | | | | |

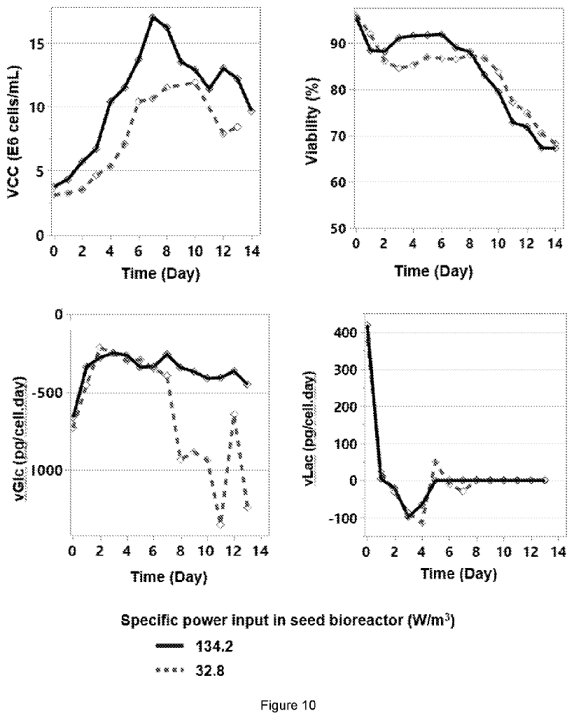

These new experiments confirmed the finding of Example 1. Indeed as shown in FIG. 10 (depicting the cell culture performances in the PBR), cell growth and product formation were lower in the production bioreactor when the SBR was cultivated with a P/V at 32.8 W/m³ associated to a higher P/V in the PBR. The lactate production and glucose consumption rates were higher in these conditions in comparison to those in which the SBR was conducted with a P/V at 134.2 W/cm3 associated to a lower P/V in the PBR.

Without being bond to any theory, it is possible that the transfer of cells from a bioreactor with stronger mechanical stress to an environment in the production bioreactor with a lower mechanical stress could still be one of the most critical factors. If cells experience more mechanical stress in the production bioreactor, they are potentially more likely to exhibit a lag in their growth or not growth at all.

### REFERENCES

1) Hecklau C et al. (2016) J Biotech, 218:53-63
2) Zang Li. et al. (2011) Anal. Chem, 83:5422-5430
3) Grammatikos et al. (1999) Biotechnol. Bioeng., 5; 64(3):357-67
4) Ozturk & Hu (2005) Cell Culture Technology for Pharmaceutical and Cell-Based Therapies, ed. CRC Press
5) Roustan M. (2005) Techniques de l'Ingénieur. Article No. J3802
6) WO98/08934
7) US2006/0148074
8) WO2015/197772

1-14. (canceled)

15. A process for producing a recombinant protein in a production bioreactor, wherein the process comprises:
(a) inoculating a N−1 bioreactor with mammalian cells comprising a gene that encodes the recombinant protein,
(b) culturing the mammalian cells in the N−1 bioreactor run in a fed-batch mode under specific conditions selected from:
(i) specific modes and duration of the feed or of at least one of the feeds,
(ii) control of the total quantity of feed(s) to be added, and/or
(iii) control of at least one engineering parameter,
(c) inoculating a N bioreactor at a seeding density of at least 2.00×10⁶ viable cells/ml with cells obtained from step (b);
(d) culturing the cells in the N bioreactor under conditions that allow production of the recombinant protein,
(e) and optionally harvesting the recombinant protein, purifying the recombinant protein and formulating the recombinant protein.

16. The process according to claim 15, wherein the feeds of step (b) are added daily, and wherein the feeds are added at day 0 of culture or later.

17. The process according to claim 15, wherein: i) at least the main feed of step (b) is added daily as a bolus, and/or ii) at least one of the secondary feeds of step (b) if any, is added daily as a bolus.

18. The process according to claim 17, wherein: i) at least the main feed of step (b) is added daily as a bolus in about 3 hours or less, and/or ii) at least one of the secondary feeds of step (b) if any, is added daily as a bolus in about 3 hours or less.

19. The process according to claim 18, wherein: i) at least the main feed of step (b) is added daily as a bolus in about 2 hours or less, and/or ii) at least one of the secondary feeds of step (b) if any, is added daily as a bolus in about 2 hours or less.

20. The process according to claim 18, wherein: i) at least the main feed of step (b) is added daily as a bolus in about 1 hour or less, and/or ii) at least one of the secondary feeds of step (b) if any, is added daily as a bolus in about 1 hour or less.

21. The process according to claim 15, wherein all the feeds of step (b) are added daily as a bolus.

22. The process according to claim 21, wherein all the feeds of step (b) are added daily as boluses in about 3 hours or less, in about 2 hours or less or in about 1 hour or less.

23. The process according to claim 15, wherein the at least one engineering parameter that is controlled during step (b) is the specific power input.

24. The process according to claim 23, wherein the specific power input is controlled daily, and wherein said specific power input in step (b) should reach at least 100 W/m³ for all or part of the duration of step (b).

25. The process according to claim 15, wherein the total quantity of feeds added during step (b) is above about 3.5%.

26. The process according to claim 25, wherein the total quantity of feeds added during step (b) is above about 3.5% but no more than 30% per culture start volume.

27. The process according to claim 15, wherein the mammalian cells are Chinese Hamster Ovary (CHO) cells.

28. The process according to claim 15, wherein the recombinant protein is a cytokine, a growth factor, a hormone, an antibody or a fusion protein.

29. The process according to claim 15, wherein the process is performed at large scale.

30. A process for improving mammalian cell growth in a production bioreactor wherein the process comprises:
(a) inoculating a N−1 bioreactor with mammalian cells comprising a gene that encodes the recombinant protein,
(b) culturing the mammalian cells in the N−1 bioreactor run in a fed-batch mode under specific conditions selected from:
(i) specific modes and duration of addition of the feed or of at least one of the feeds,
(ii) control of the total quantity of feed(s) to be added, and/or
(iii) control of at least one engineering parameter,
(c) inoculating a N bioreactor at a seeding density of at least 2.00×10⁶ viable cells/ml with cells obtained from step (b);
(d) culturing the cells in the N bioreactor under conditions that allow production of the recombinant protein,
(e) and optionally harvesting the recombinant protein, purifying the recombinant protein and formulating the recombinant protein.

31. A process for increasing the yield of production of a recombinant protein expressed by mammalian cells in culture in a production bioreactor, wherein the process comprises:
(a) inoculating a N−1 bioreactor with mammalian cells comprising a gene that encodes the recombinant protein,
(b) culturing the mammalian cells in the N−1 bioreactor run in a fed-batch mode under specific conditions selected from:
(i) specific modes and duration of the feed or of at least one of the feeds,
(ii) control of the total quantity of feed(s) to be added, and/or
(iii) control of at least one engineering parameter,
(c) inoculating a N bioreactor at a seeding density of at least 2.00×10⁶ viable cells/ml with cells obtained from step (b);
(d) culturing the cells in the N bioreactor under conditions that allow production of the recombinant protein,
(e) and optionally harvesting the recombinant protein, purifying the recombinant protein and formulating the recombinant protein.
